(19) United States
(12) Patent Application Publication
(10) Pub. No.: US 2023/0148870 A1
(43) Pub. Date: May 18, 2023

(54) MULTI-OBJECT THERMAL RADIATION MEASURING DEVICE AND OPERATING METHOD THEREOF

(71) Applicant: Electronics and Telecommunications Research Institute, Daejeon (KR)

(72) Inventors: Jae Young JUNG, Daejeon (KR); You Jin KIM, Daejeon (KR); Won Kyu CHOI, Daejeon (KR); Se Han KIM, Daejeon (KR)

(73) Assignee: Electronics and Telecommunications Research Institute, Daejeon (KR)

(21) Appl. No.: 17/892,718

(22) Filed: Aug. 22, 2022

(30) Foreign Application Priority Data
Nov. 15, 2021 (KR) 10-2021-0156847

(51) Int. Cl. A61B 5/01; A61B 5/00

(52) U.S. Cl. CPC A61B 5/01; A61B 5/0075; A61B 5/7267; A61B 5/7275; A61B 5/746; A61B 5/6889; A61B 5/0008; A61B 2560/0233

(57) ABSTRACT

A multi-object thermal radiation measuring device may include: a sensing unit including a thermal sensor, and configured to generate a thermal image for a measurement target space based on a detection signal of the thermal sensor; a multi-object thermal radiation measuring unit configured to detect a specific portion of a measurement target object by applying an image recognition technology to the thermal image, and acquire and store a thermal radiation measurement value of the specific portion; a disease symptom detection unit configured to determine whether each measurement target object is abnormal, and detect a disease symptom of an abnormal object based on a result obtained by precisely measuring the thermal radiation of the abnormal object; and a driving unit configured to rotate the sensing unit according to a command transferred by any one of the multi-object thermal radiation measuring unit and the disease symptom detection unit.

### CROSS-REFERENCE TO RELATED APPLICATION

This application claims priority to and the benefit of Korean Patent Application No. 10-2021-0156847, filed on Nov. 15, 2021, the disclosure of which is incorporated herein by reference in its entirety.

### BACKGROUND

### 1. Technical Field

The present disclosure relates to a thermal radiation measuring device and an operating method thereof.

### 2. Related Art

Recently, high-risk high-fatality respiratory infectious diseases such as COVID-19, MERS, and SARS, which are highly contagious, have been spread around the globe, and thus paralyzed the normal social function and caused enormous social, economic, and human damage. The above-described respiratory infectious diseases usually occur in a concentrated/sealed/crowded space, and it is more important than anything to block the spread of the respiratory infectious diseases in advance through an early diagnosis for the symptoms of multiple objects in an indoor space. The symptoms of the respiratory infectious diseases are determined through early signs such as fever, cough, sneeze, and respiratory difficulty. Such early signs need to be constantly monitored.

Conventionally, a thermal camera which can measure body temperature remotely in a non-contact manner has been mainly used to sense the early signs of the respiratory infectious diseases. However, when the body temperatures of people who pass in an airport, school, hospital, or bus terminal are measured through a thermal camera installed in a waiting room on one side of the corresponding place, the measurement accuracy for skin temperatures may be degraded by the influence of outside temperature, and it is highly likely to miss a patient with fever because the sensitivity of the thermal camera is low. Furthermore, it is impossible to constantly monitor the body temperatures of many people in a concentrated/sealed/crowded space through the conventional method.

Besides, the conventional thermal camera has no function of analyzing symptoms such as cough and sneeze, which are accompanied by a fever, and thus is inadequate to preemptively deal with the spread of infections.

### SUMMARY

Various embodiments are directed to an unmanned automated thermal radiation measuring device which can preemptively deal with the spread of infections by regularly and continuously monitoring the infection risk of a concentrated/sealed/crowded space, and an operating method thereof. Specifically, various embodiments are directed to a thermal radiation measuring device which can detect respiratory disease symptoms of multiple objects in a concentrated/sealed/crowded space at the early stage, centrally manage abnormal object thermal radiation measurement values and abnormal object behavior detection result values through a cloud-based service platform based on ICT technology, and notice the disease symptoms and the disease risk to the whole nation, and an operating method thereof.

The objects of the present disclosure are not limited to the above-mentioned objects, and the other objects which are not mentioned herein will be clearly understood from the following descriptions by those skilled in the art.

In an embodiment, a multi-object thermal radiation measuring device may include: a sensing unit including a thermal sensor, and configured to generate a thermal image for a measurement target space based on a detection signal of the thermal sensor; a multi-object thermal radiation measuring unit configured to detect a specific portion of a measurement target object by applying an image recognition technology to the thermal image, and acquire and store a thermal radiation measurement value of the specific portion; a disease symptom detection unit configured to determine whether each measurement target object is abnormal, according to a predetermined criterion based on the thermal radiation measurement value, and detect a disease symptom of an abnormal object based on a result obtained by precisely measuring the thermal radiation of the abnormal object; and a driving unit configured to rotate the sensing unit according to a command transferred by any one of the multi-object thermal radiation measuring unit and the disease symptom detection unit.

The sensing unit may be installed on the ceiling of the measurement target space.

The sensing unit may include a distance sensor, and measure a distance to the measurement target object through the distance sensor, and the multi-object thermal radiation measuring unit may compensate for the thermal radiation measurement value by applying a correction value that is predetermined according to the distance to the measurement target object.

The multi-object thermal radiation measuring device may further include a sensor calibration unit configured to calibrate a sensor included in the sensing unit. The sensing unit may include a distance sensor, the sensor calibration unit may be installed at a predetermined distance from the sensing unit, and calibrates the sensor by using a blackbody whose temperature is variably set by the sensor calibration unit, and the driving unit may rotate the sensing unit according to a command of the sensor calibration unit, such that the sensing unit faces the blackbody.

The sensing unit and the blackbody may be installed on the ceiling of the measurement target space.

The multi-object thermal radiation measuring device may further include: an object detection unit configured to detect the measurement target object by applying an image recognition technology to the thermal image, and calculate the distribution of measurement target objects according to a measurement direction; and a dynamic switching unit configured to determine a thermal radiation measurement time for each measurement section based on the distribution. The multi-object thermal radiation measuring unit may generate a command for rotation of the sensing unit based on the thermal radiation measurement time for each measurement section, the driving unit may rotate the sensing unit according to the command of the object detection unit, and the measurement section may be configured by dividing the measurement target space according to a predetermined criterion based on the rotatable range of the sensing unit.

The specific portion may be at least any one of the forehead and inner canthus of the measurement target object.

The disease symptom detection unit may extract a spatial coordinate of the abnormal object from a thermal image in which the abnormal object appears, generate a command for rotation of the sensing unit based on the spatial coordinate and a tracking result for the abnormal object, transfer the command to the driving unit, receive, from the sensing unit, an additional thermal image in which the abnormal object appears, and precisely measure the thermal radiation of the abnormal object based on the additional thermal image.

The disease symptom detection unit may detect the behavior of the abnormal object by utilizing a deep learning-based behavior detection model, and detect a disease symptom of the abnormal object based on at least any one of the thermal radiation precise measurement result and the behavior detection result.

The multi-object thermal radiation measuring device may further include a communication unit. The disease symptom detection unit may generate metadata on the thermal radiation precise measurement value, and the communication unit may transmit the metadata to an information system which is present outside.

In an embodiment, a disease risk warning system may include: the multi-object thermal radiation measuring device, and a disease symptom early detection platform server configured to calculate a disease risk based on the disease symptom detection result and the metadata collected by the multi-object thermal radiation measuring device, and provide an alarm service for the disease risk by using at least any one communication method of a wired communication method and a wireless communication method.

In an embodiment, an operating method of a multi-object thermal radiation measuring device may include: a multi-object thermal radiation measuring step of detecting a specific portion of a measurement target object by applying an image recognition technology to a thermal image, and acquiring and storing a thermal radiation measurement value of the specific portion; an abnormal object determination step of determining whether each measurement target object is abnormal, according to a predetermined criterion based on the thermal radiation measurement value; an abnormal object thermal radiation precise measurement step of precisely measuring the thermal radiation of an abnormal object based on a thermal image in which the abnormal object appears; and an abnormal object information transmitting step of detecting a disease symptom of the abnormal object based on the thermal radiation measurement result, and transmitting metadata on the thermal radiation precise measurement value and the disease symptom detection result to a disease symptom early detection platform server.

The operating method may further include a sensor calibration step of calibrating a sensor included in the multi-object thermal radiation measuring device, before the multi-object thermal radiation measuring step.

The operating method may further include, before the multi-object thermal radiation measuring step: an object detection step of detecting the measurement target object by applying the image recognition technology to the thermal image, and calculating the distribution of measurement target objects according to a measurement direction; and an inventory switching time calculation step of calculating a thermal radiation measurement time for each measurement section based on the distribution.

The operating method may further include, after the multi-object thermal radiation measuring step, a thermal radiation measurement value compensation step of compensating for the thermal radiation measurement value by applying a correction value that is predetermined according to a distance to the measurement target object.

The operating method may further include, after the abnormal object thermal radiation precise measurement step, an abnormal object behavior detection step of detecting the behavior of the abnormal object by utilizing a deep learning-based behavior detection model.

The operating method may further include, after the abnormal object information transmitting step, an alarm service step of calculating a disease risk based on the metadata and the disease symptom detection result, and providing an alarm service for the disease risk by using at least any one communication method of a wired communication method and a wireless communication method.

The specific portion of the measurement target object may be at least any one of the forehead and inner canthus of the measurement target object.

The sensor calibration step may include calibrating the sensor by using a blackbody whose temperature is variably set.

The abnormal object behavior detection step may include detecting whether the abnormal object has at least any one of cough and sneeze, by utilizing a deep learning-based behavior detection model.

In accordance with the embodiments of the present disclosure, a single thermal radiation measuring device may be used to continuously, precisely, and efficiently measure thermal radiations of multiple objects in a concentrated/sealed/crowded space, which makes it possible to significantly reduce the installation cost of the measuring device. Furthermore, disease symptoms such as cough and sneeze may be detected through the artificial intelligence technology, which makes it possible to raise the accuracy of disease symptom early detection. Finally, when multi-object thermal radiation measuring devices installed over the country transmit object thermal radiation measurement values and abnormal object behavior detection result values to a cloud-based service platform, the platform performs data monitoring, risk evaluation, service management and the like, and provide an alarm service to notice the disease symptoms and the disease risk to the whole nation through nationwide networks at the early stage.

The effects obtainable by the present disclosure are not limited to the above-mentioned effects, and the other effects which are not mentioned herein will be clearly understood from the following descriptions by those skilled in the art.

### BRIEF DESCRIPTION OF THE DRAWINGS

FIG. 1 is a diagram illustrating the configuration of a conventional thermal camera.

FIGS. 2A to 2C are diagrams illustrating an operating method of the conventional thermal camera.

FIG. 3 is a diagram illustrating an operating method of a multi-object thermal radiation measuring device in accordance with an embodiment of the present disclosure.

FIGS. 4A and 4B are diagrams for describing the operating method of the multi-object thermal radiation measuring device that dynamically optimizes inventory switching times, in accordance with the embodiment of the present disclosure.

FIG. 5 is a block diagram illustrating the configuration of the multi-object thermal radiation measuring device in accordance with the embodiment of the present disclosure.

FIGS. 6A and 6B are diagrams illustrating an RGB spectrum and an infrared spectrum around the inner canthi of an object, detected by the multi-object thermal radiation measuring device in accordance with the embodiment of the present disclosure.

FIG. 7 is a diagram illustrating an example in which the multi-object thermal radiation measuring device in accordance with the embodiment of the present disclosure is installed and operated in a concentrated/sealed/crowded space.

FIG. 8 is a flowchart illustrating an operating method of the multi-object thermal radiation measuring device in accordance with the embodiment of the present disclosure.

### DETAILED DESCRIPTION

The advantages and characteristics of the present disclosure and a method for achieving the advantages and characteristics will be clearly understood through embodiments to be described below in detail with reference to the accompanying drawings. However, the present disclosure is not limited to the embodiments set forth herein, but may be embodied in different forms. Rather, these embodiments are provided so that this disclosure will be thorough and complete, and will fully convey the scope of the present disclosure to those skilled in the art. The present disclosure is only defined by the scope of claims. Terms used in this specification are used for describing embodiments while not limiting the present disclosure. The terms of a singular form may include plural forms unless specifically mentioned in phrases. The term such as “comprise” or “comprising” used in the specification specifies a mentioned component, step, operation and/or element, but does not exclude the presence or addition of other components, steps, operations and/or elements.

Moreover, detailed descriptions for publicly known technologies related to the present disclosure will be ruled out in order not to unnecessarily obscure the subject matter of the present disclosure.

The present disclosure relates to a thermal radiation measuring device which detects the disease symptoms of multiple objects in a concentrated/sealed/crowded space in advance through ICT technology, and provides the risk of the disease symptoms throughout the country while the detection results are centrally managed by a cloud-based service platform, and an operating method thereof. The multi-object thermal radiation measuring device and the operating method thereof in accordance with the embodiment of the present disclosure are mainly applied to a concentrated/sealed/crowded space which is a measurement target space. However, the scope of the present disclosure is not limited thereto, but the multi-object thermal radiation measuring device and the operating method thereof in accordance with the embodiment of the present disclosure may be applied to various types of spaces.

The multi-object thermal radiation measuring device in accordance with the present disclosure is installed on the ceiling of a concentrated/sealed/crowded space, and consecutively measures the thermal radiations of multiple objects while rotated in a top-to-bottom direction and a side-to-side direction through a motion controller. Therefore, since the multi-object thermal radiation measuring device in accordance with the present disclosure can measure the thermal radiations of many people at the same time, it is possible to reduce the installation cost of the measuring device.

Furthermore, the multi-object thermal radiation measuring device in accordance with the present disclosure can detect/track objects from an image obtained by a thermal camera (thermal sensor) through deep learning-based image recognition technology, and calculate the optimal inventory switching time (time allocated to data collection for each measurement section) according to object distributions, thereby efficiently collecting data.

Furthermore, the multi-object thermal radiation measuring device in accordance with the present disclosure may correct a sensor by using a blackbody whose temperature can be variably set, and thus secure high temperature/distance measurement accuracy while automatically compensating for an environmental error without user intervention.

Furthermore, the multi-object thermal radiation measuring device in accordance with the present disclosure may detect a region in which an object with a high fever symptom, i.e. an abnormal object, is present, and perform precise thermal radiation measurement and object behavior detection on the corresponding region by utilizing a deep learning-based behavior detection model (e.g. a cough/sneeze recognition model), thereby accurately detecting the disease symptoms. Furthermore, when the thermal radiation measuring devices installed across the country transmit, to a disease symptom early detection platform server, the disease symptom detection results and metadata of the thermal radiation precise measurement values and the abnormal object behavior detection result values, the platform server may analyze the disease risk based on the thermal radiation precise measurement values and the abnormal object behavior detection result values, and notice the disease symptoms and the disease risk to the whole nation, thereby supporting early blocking of the spread of the disease.

Hereafter, embodiments of the present disclosure will be described in detail with reference to the accompanying drawings. In order to promote understandings of the present disclosure, like components will be represented by the same reference numerals regardless of the figure numbers.

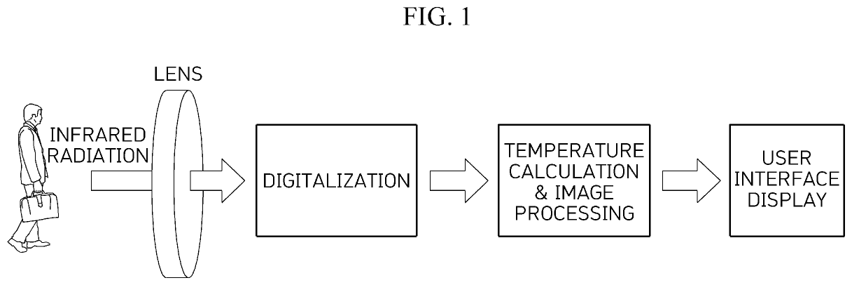

FIG. 1 is a diagram illustrating the configuration of a conventional thermal camera. As illustrated in FIG. 1, the thermal camera includes a lens for forming the focus of incident infrared light on a sensor surface, and a device and software for processing a detection signal into an image and displaying the image. The thermal camera includes a sensor which is made of various materials sensitive to infrared light wavelengths unlike a CCD (Charge Coupled Device), and has an FPA (Focal Plane Array) composed of pixels each having a micrometer size. In general, a microbolometer is used as a detector of the thermal camera. When the temperature of a target object changes, the temperature of the microbolometer changes, and the change is converted into an electrical signal, which makes a thermal image.

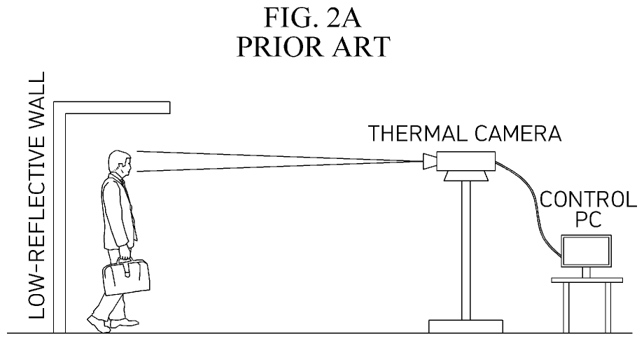

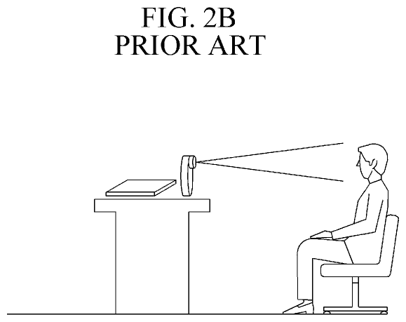

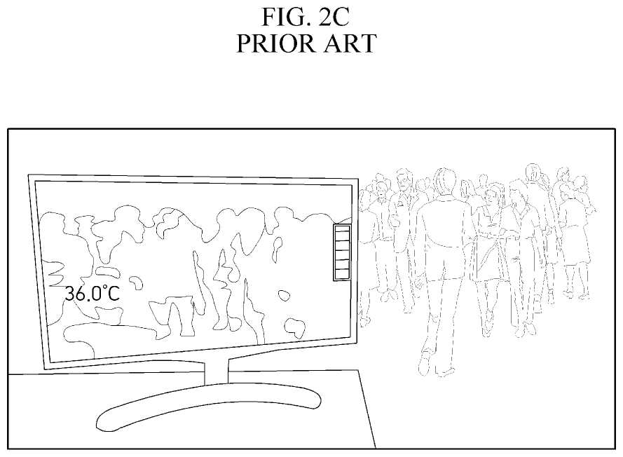

FIGS. 2A to 2C are diagrams illustrating an operating method of the conventional thermal camera. The operating method of the conventional thermal camera will be described as follows. As illustrated in FIGS. 2A and 2B, a user 51 of the thermal camera fixedly installs the thermal camera to a specific position, and measures the temperature of the skin (generally, forehead) of a subject while the subject stands or sits at a designated position spaced by a predetermined distance apart from the installation position of the camera. As illustrated in FIG. 2A, a PC for controlling the thermal camera and a low-reflective wall for measurement accuracy may be additionally provided. However, when the body temperature (thermal radiation) is measured through the method illustrated in FIGS. 2A and 2B, it is difficult to precisely measure the body temperature because the distance between the subject and the thermal camera and the measurement height are not constant, and to measure the body temperatures of many people at the same time.

As illustrated in FIG. 2C, when the body temperatures of people who pass in an airport, school, hospital, or bus terminal are measured through a thermal camera installed in a waiting room on one side of the corresponding place, the measurement accuracy for skin temperatures is degraded by the influence of outside temperature, and a measurement value error occurs depending on the distance between the thermal camera and an object (e.g. human). For reference, the thermal camera detects thermal temperature information by converting infrared light energy radiated from a measurement target into a current or voltage. Thus, as the measurement distance increases, energy absorbed by the thermal camera decreases while a temperature deviation increases.

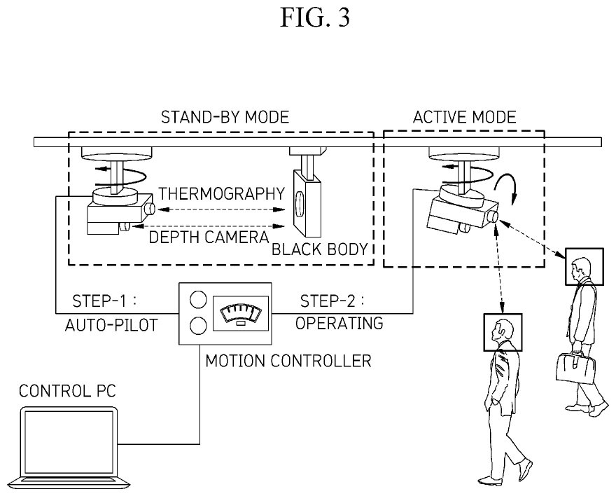

FIG. 3 is a diagram illustrating an operating method of a multi-object thermal radiation measuring device 10 in accordance with an embodiment of the present disclosure. Unlike the operating method of the conventional thermal camera, a sensing unit 700 of the multi-object thermal radiation measuring device 10 in accordance with the embodiment of the present disclosure is installed on the ceiling of a concentrated/sealed/crowded space, and consecutively measures the thermal radiations of multiple objects while moved or rotated in the top-to-bottom direction and the side-to-side direction under control of a motion controller 610.

First, a stand-by mode in which an auto-pilot operation is performed as a first step is a mode before the thermal radiations of the multiple objects in the concentrated/sealed/crowded space are measured. In the stand-by mode, a temperature sensor and a distance sensor, which are included in the thermal radiation measuring device, are automatically calibrated. That is, in the stand-by mode, the sensors included in the multi-object thermal radiation measuring device 10 are auto-tuned without a re-tuning process, when a monitoring result for the environment around the concentrated/sealed/crowded space indicates that an environmental change is detected. The stand-by mode is a mode for supporting the multi-object thermal radiation measuring device 10 to operate at the best performance at all times. The stand-by mode may enable a user to install the thermal radiation measuring device without an expert's help.

In an active mode in which an operation of the multi-object thermal radiation measuring device 10 is performed as a second step, the multi-object thermal radiation measuring device 10 (or the sensing unit 700 of the multi-object thermal radiation measuring device 10) that is attached to the ceiling of the concentrated/sealed/crowded space and coupled to the motion controller 610 measures the thermal radiations of the multiple objects in the concentrated/sealed/crowded space while rotated in the top-to-bottom direction and the side-to-side direction. When the thermal radiation measurement result values are abnormal, the multi-object thermal radiation measuring device 10 detects a region in which an abnormal object is present, precisely and continuously monitors the behaviors of the abnormal object such as cough and sneeze in the region, and thus detects the symptoms of a respiratory infectious disease at the early stage.

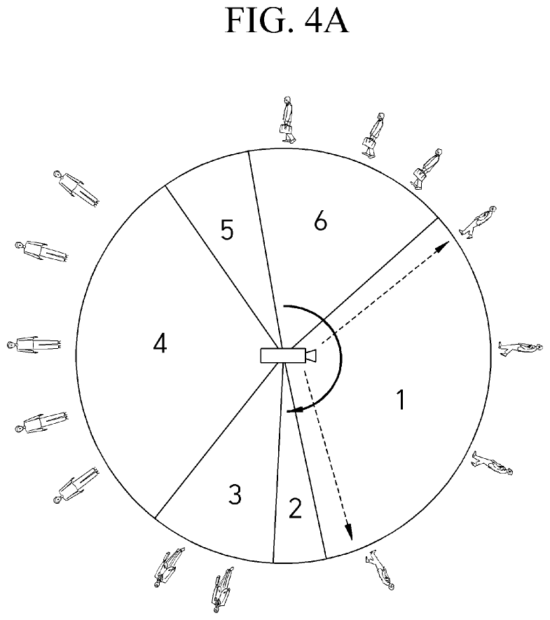

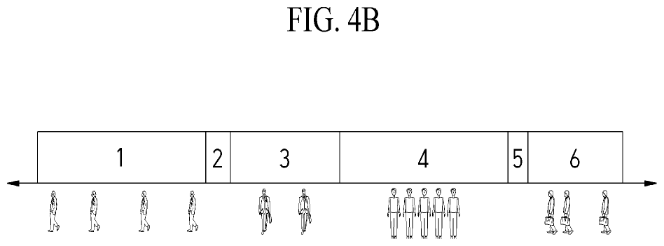

FIGS. 4A and 4B are diagrams for describing the operating method of the multi-object thermal radiation measuring device 10 that dynamically optimizes inventory switching times, in accordance with the embodiment of the present disclosure. FIG. 4A illustrates measurement target spaces (measurement sections) which are divided according to the rotation angle range of the sensing unit 700 of the multi-object thermal radiation measuring device 10, and FIG. 4B illustrates inventory switching times allocated to the respective measurement sections along the time axis. The inventory switching time refers to a time that is allocated to each of the measurement sections for the multi-object thermal radiation measuring device 10 to collect data (data collection time for each measurement section). The measurement target space of the multi-object thermal radiation measuring device 10 may be divided into a plurality of measurement sections based on the rotatable range of the sensing unit 700 or the direction of the sensing unit 700. For example, when the initial direction immediately before the operation of the sensing unit 700 is set to 0°, the entire space may be divided into three measurement sections of 0° to 120°, 120° to 240°, and 240° to 360° based on the horizontal rotation angles. The thermal radiation measuring device 10 optimizes the inventory switching times based on auto-detection results for the objects. That is, the thermal radiation measuring device 10 may set the inventory switching time of a section in which the number of objects exceeds a predetermined reference value to a longer inventory switching time than that of a section in which the number of objects falls within the reference value, based on a result obtained by auto-scanning the concentrated/sealed/crowded space. As such, the multi-object thermal radiation measuring device 10 may distribute different inventory switching times to the respective measurement sections according to the distribution of the measurement target objects, in order to efficiently perform the measurement. As illustrated in FIGS. 4A and 4B, a longer inventory switching time is allocated to measurement sections ‘1’ and ‘4’, in which the numbers of objects are relatively high, than a measurement section ‘2’ or ‘5’ in which the number of objects is 0. That is, the multi-object thermal radiation measuring device 10 allocates a short inventory switching time to a measurement section having a small number of objects, and allocates a long inventory switching time to a measurement section having a large number of objects, such that thermal radiation measurement can be more precisely performed in the measurement section having a large number of objects than in the measurement section having a small number of objects.

The measurement sections may be set by evenly dividing the measurement target space based on the angle range in which the sensing unit can be rotated in the top-to-bottom direction or the side-to-side direction. Furthermore, the angle ranges of the measurement sections may be differently set depending on the characteristics of the spaces. For example, the angle range of a measurement section in which people rarely gather may be set to a large range, and the angle range of a measurement section in which many people gather at all times may be set to a small range. In the case of a space in which the distribution of objects is constant, the inventory switching time for each section may be set to a specific value, instead of dynamically optimizing the inventory switching time.

In accordance with another aspect of the present disclosure, the multi-object thermal radiation measuring device 10 may apply different rotational angular velocities to the respective sections, instead of the inventory switching times. That is, the multi-object thermal radiation measuring device 10 may apply different rotational angular velocities to the respective measurement sections depending on the object distribution. For example, the multi-object thermal radiation measuring device 10 may lower the rotational angular velocity for a measurement section in which objects are crowded, and raise the rotational angular velocity for a measurement section in which no objects are present.

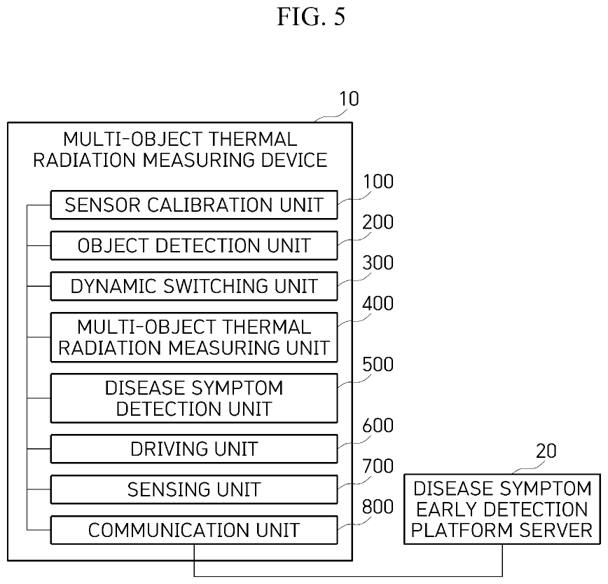

FIG. 5 is a block diagram illustrating the configuration of the multi-object thermal radiation measuring device 10 in accordance with the embodiment of the present disclosure. The multi-object thermal radiation measuring device 10 in accordance with the embodiment of the present disclosure may include a sensor calibration unit 100, an object detection unit 200, a dynamic switching unit 300, a multi-object thermal radiation measuring unit 400, a disease symptom detection unit 500, a driving unit 600, the sensing unit 700, and a communication unit 800.

The multi-object thermal radiation measuring device 10 in accordance with the embodiment of the present disclosure is switched to the stand-by mode when power is supplied thereto. At this time, the sensor calibration unit 100 is operated.

The sensor calibration unit 100 calibrates sensors included in the sensing unit 700. For example, the sensor calibration unit 100 calibrates a temperature sensor (e.g. thermal sensor) and a distance sensor (e.g. depth sensor) which are included in the sensing unit 700. Since the sensors may be degraded in performance with the elapse of time, it is necessary to periodically calibrate the sensors in order to reduce measurement errors. Furthermore, the sensor calibration unit 100 monitors the environment around the concentrated/sealed/crowded space, and performs the initial setting operation (sensor calibration) when the environment is changed. The sensor calibration unit 100 serves to support the multi-object thermal radiation measuring device 10 to operate at the best performance at all times. The sensor calibration unit 100 automatically calibrates the sensors of the multi-object thermal radiation measuring device 10 such that a user manipulation process such as a process of re-tuning the thermal radiation measuring device is not needed even though the environment around the concentrated/sealed/crowded space is changed. The sensor calibration unit 100 includes a temperature sensor calibration module 110 and a distance sensor calibration module 120.

The temperature sensor calibration module 110 calibrates the temperature sensor (e.g. thermal sensor) included in the sensing unit 700 (temperature calibration). The temperature sensor calibration performed by the temperature sensor calibration module 110 will be described as follows. Some of commercially available thermal cameras calibrate a sensor through a blackbody 130 before measuring the thermal radiations of objects, in order to raise the accuracy of temperature measurement by reducing errors caused by the temperature measurement environment. The blackbody refers to an object that theoretically only absorbs external energy and does not reflect the external energy, and has a radiation factor defined as ‘1’. In existing thermal cameras, the blackbody needs to be installed adjacent to a target object, and operates through only one initial calibration. Therefore, when the existing thermal radiation cameras are continuously operated, measurement errors may occur. In the present disclosure, however, the blackbody 130 is installed on the ceiling of the concentrated/sealed/crowded space. Thus, the temperature sensor of the thermal radiation measuring device may be calibrated without disturbing the target object. However, only when the blackbody 130 is fixed installed at a predetermined distance from the sensing unit 700, it is possible to precisely calibrate the sensor. Furthermore, the sensor calibration unit 100 in accordance with the present disclosure may variably set the temperature of the blackbody 130 through wired/wireless communication with the blackbody 130. In another embodiment, the communication with the blackbody 130 may be performed by the communication unit 800. In the conventional thermal radiation measuring device, the temperature sensor calibration is performed only for the blackbody having one specific temperature. However, the blackbody 130 in accordance with the present disclosure has a variable temperature range of 34 to 39° C., which is recommended by FDA (Food and Drug Administration), and the temperature of the blackbody 130 may be set to a random temperature within the corresponding range. The temperature sensor calibration module 110 in accordance with the embodiment of the present disclosure can calibrate the temperature sensor (e.g. thermal sensor) at a random temperature in the range of 34 to 39° C. Through this process, the multi-object thermal radiation measuring device 10 may raise the accuracy of the temperature measurement.

In accordance with another aspect of the present disclosure, when the sensing unit 700 is attached or connected to a portion of a structure installed in the measurement target space, not the ceiling of the measurement target space, the blackbody 130 may not be attached to the ceiling of the measurement target space, but be installed at a predetermined distance from the sensing unit 700 through another method in which the blackbody 130 is attached or connected to the structure, for example.

The distance sensor calibration (distance calibration) performed by the distance sensor calibration module 120 will be described as follows. The thermal camera detects infrared energy radiated by a measurement target, and converts the detected energy into temperature information. When the measurement distance increases, the amount of energy absorbed by the thermal camera decreases, and a temperature deviation increases. Thus, it is necessary to accurately measure the distance from the measurement target. Therefore, the distance sensor needs to be calibrated. The sensing unit 700 of the thermal radiation measuring device 10 includes a thermal sensor, an RGB sensor, and a depth sensor (or depth camera), and the distance sensor calibration module 120 measures the distance to the blackbody whose location is fixed, through the depth sensor, and then calibrates the depth sensor by using the measurement value. Through such a distance calibration method, the distance to the measurement target object may be precisely measured. The depth sensor may be replaced with a radar sensor or lidar sensor.

The sensor calibration unit 100 transfers a command to the driving unit 600, and the driving unit 600 rotates the sensing unit 700 according to the command, such that the sensing unit 700 faces the blackbody. Then, the sensor calibration unit 100 performs sensor calibration.

The object detection unit 200 applies an image recognition technology to an image received from the sensing unit 700, auto-detects a measurement target object that appears in the corresponding image, determines whether the object is present, and calculates object distribution. The image which the sensing unit 700 transfers to the object detection unit 200 may be a thermal spectrum image (hereafter, referred to as ‘thermal image’) or RGB spectrum image (hereafter, ‘RGB image’). The sensing unit 700 may transfer distance information for each measurement target object to the object detection unit 200. The object detection unit 200 performs object detection or object tracking by applying a deep learning model to the thermal image or RGB image. The object detection unit 200 may not only detect and track one object, but also detect and track a plurality of objects (multiple objects) at the same time. The object detection unit 200 may calculate object distribution (the distribution of the numbers of objects) according to measurement directions, based on the object detection result. In order to detect the measurement target object, the object detection unit 200 may transfer a command to the driving unit 600, such that the driving unit 600 rotates the sensing unit 700 according to the command, and the sensing unit 700 may generate a thermal image or RGB image while rotated, and transfers the generated image to the object detection unit 200.

The dynamic switching unit 300 calculates inventory switching times for the respective measurement sections of the concentrated/sealed/crowded space (thermal radiation measurement times for the respective measurement sections) based on the object distribution or the object detection result (object scan result) of the object detection unit 200. In this case, the dynamic switching unit 300 dynamically optimizes the inventory switching times according to the object distribution within the space. The method in which the dynamic switching unit 300 optimizes the inventory switching times has been described above with reference to FIGS. 4A and 4B.

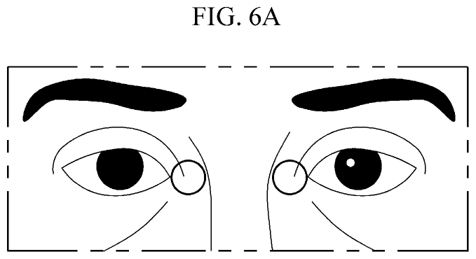

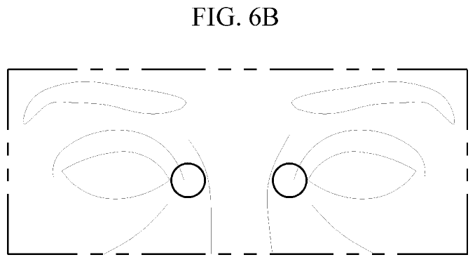

The multi-object thermal radiation measuring unit 400 measures the thermal radiations of the multiple objects according to the inventory switching times (thermal radiation measurement time for each measurement section) set by the dynamic switching unit 300. Furthermore, the multi-object thermal radiation measuring unit 400 may compensate for the thermal radiation measurement value of a measurement target object by applying a correction value that is predetermined according to the distance to the measurement target object. The multi-object thermal radiation measuring unit 400 generates a command for rotation of the sensing unit 700 based on the inventory switching times for the respective measurement sections, and transfers the generated command to the driving unit 600. The command for rotation of the sensing unit 700 may include a rotation angle and a rotation time. The rotation angle may be replaced with a target direction, and the rotation time may be replaced with a rotational angular velocity. The driving unit 600 receives the rotation command from the multi-object thermal radiation measuring unit 400, and rotates the sensing unit 700 in the top-to-bottom direction and the side-to-side direction through the motion controller 610. When the multi-object thermal radiation measuring device 10 is configured with the all-in-one design, the driving unit 600 rotates the multi-object thermal radiation measuring device 10 in the top-to-bottom direction and the side-to-side direction according to the rotation command. The sensing unit 700 generates a thermal image and an RGB image during the rotation, and transfers the generated images to the multi-object thermal radiation measuring unit 400. The multi-object thermal radiation measuring unit 400 detects the face, forehead, and inner canthus of an object in the thermal image and the RGB image by applying the image recognition technology to the images, and acquires, processes, and stores thermal radiation measurement values of the corresponding portions. The position of the inner canthus is marked as a circle in FIGS. 6A and 6B. FIG. 6A shows an RGB spectrum around the inner canthus of an object, detected by the multi-object thermal radiation measuring device, and FIG. 6B illustrates an infrared spectrum around the inner canthus of an object, detected by the multi-object thermal radiation measuring device. For reference, a plurality of documents show that the inner canthus is the most suitable position for sensing a fever, because the inner canthus has thin skin, is less exposed to the environment, and is located right above the main artery. At this time, the face, forehead, and inner canthus of the object may be detected by any one of the thermal sensor and the RGB sensor, or a combination of the two sensors. The sensing unit 700 collects distance data between the distance sensors (e.g. depth sensor, radar sensor, and lidar sensor) and the measurement target object, and transfers the collected distance data to the multi-object thermal radiation measuring unit 400.

A thermal radiation measurement value compensation module 410 included in the multi-object thermal radiation measuring unit 400 compensates for the thermal radiation measurement value based on the distance data between the distance sensor of the sensing unit 700 and the object (or the forehead or inner canthus of the object). That is, the thermal radiation measurement value compensation module 410 compensates for the thermal radiation measurement value by applying a correction value that is predetermined according to the distance. As the distance between the temperature sensor (e.g. thermal sensor) and the object increases, temperature measurement values of objects (targets) having the same size generally decrease. Thus, the measurement values needs to be compensated for. For example, the thermal radiation measurement value compensation module 410 may set a reference distance to 2 m, and compensate for the thermal radiation measurement value by adding one degree to the thermal radiation measurement value when the distance to the object is 3 m, or adding two degrees to the thermal radiation measurement value when the distance to the object is 4 m.

The disease symptom detection unit 500 determines whether the objects are normal, based on the multi-object thermal radiation measurement results (the thermal radiation measurement values), and then precisely measure the thermal radiation of an object having an abnormal state (hereafter, referred to as ‘abnormal object’) and detects the behavior of the abnormal object, thereby detecting a disease symptom. The disease symptom detection unit 500 detects (determines) the disease symptom of the abnormal object based on at least any one of the thermal radiation precise measurement result for the abnormal object and the behavior detection result for the abnormal object.

The disease symptom detection unit 500 uses a thermal image in which the abnormal object appears, in order to precisely measure the thermal radiation of the abnormal object. Furthermore, the disease symptom detection unit 500 uses the thermal image or RGB image in which the abnormal object appears, in order to detect the behavior of the abnormal object. The disease symptom detection unit 500 includes an object state determination module 510, an abnormal object region detection module 520, an abnormal object thermal radiation precise measurement module 530, and an abnormal object behavior detection module 540. The object state determination module 510 determines whether the objects are normal, based on the multi-object thermal radiation measurement results generated by the multi-object thermal radiation measuring unit 400. When the determination result indicates that the objects are normal, the multi-object thermal radiation measuring unit 400 consecutively measures the thermal radiations of the objects in the concentrated/sealed/crowded space. When the determination result indicates that an object is abnormal (e.g. high fever symptom), the abnormal object region detection module 520 detects a region in which the abnormal object is present, through the image recognition technology. Then, the abnormal object thermal radiation precise measurement module 530 precisely measures the thermal radiation of the abnormal object. The abnormal object thermal radiation precise measurement module 530 may extract the spatial coordinate of the abnormal object or the abnormal object region (the space range in which the abnormal object is present) from the thermal image in which the abnormal object appears, generate a command for rotation of the sensing unit 700 based on the spatial coordinate (or abnormal object region) and the abnormal object tracking result, transfer the command to the driving unit 600, collect additional thermal images in which the abnormal object appears from the sensing unit 700, and precisely measure the thermal radiation of the abnormal object based on the collected additional thermal images.

Furthermore, the abnormal object behavior detection module 540 detects the behavior of the abnormal object based on the abnormal object region detection result. For example, the abnormal object behavior detection module 540 detects the behavior of the abnormal object, such as cough, sneeze, or respiratory difficulty, from the thermal image or the RGB image in which the abnormal object appears. The abnormal object behavior detection module 540 may utilize a deep learning model in order to detect the disease symptoms of the abnormal object at the early stage. For example, the abnormal object behavior detection module 540 tracks the location of an object who coughs or sneezes or the number of times that the object coughs or sneezes, by using a cough/sneeze recognition model (behavior detection model) generated through a supervised learning technique based on a CNN (Convolutional Neural Network), in order to detect the disease symptom of the abnormal object.

The abnormal object thermal radiation precise measurement module 530 generates metadata on the abnormal object thermal radiation precise measurement value, for example, the maximum value, average value, and minimum value of the temperature of the corresponding abnormal object, and the abnormal object behavior detection module 540 generates metadata on the abnormal object behavior detection result value, for example, the average number of times that the abnormal object coughs per minute. The disease symptom detection unit 500 transmits the metadata on the abnormal object thermal radiation measurement value, the metadata on the abnormal object behavior detection result value, and the disease symptom detection result of the abnormal object to a disease symptom early detection platform server 20 through the communication unit 800, such that the metadata and the detection result are managed by the disease symptom early detection platform server 20. The disease symptom early detection platform server 20 is a cloud-based service platform, and monitors data collected by the multi-object thermal radiation measuring devices 10 installed in main concentrated/sealed/crowded spaces across the country, evaluates the disease risk, and performs service management, thereby detecting the disease symptoms at the early stage, and providing an alarm service to notice the disease risk to the whole nation. Specifically, the disease symptom early detection platform server 20 may calculate the disease risk based on the metadata on thermal radiation precise measurement values collected through nationwide networks, the metadata on the abnormal object behavior detection result values, and the disease symptom detection result of the abnormal object, and provide an alarm service to inform the whole nation of the disease risk and the statistics of the disease symptom detection results through at least any one communication method of a wired communication method and a wireless communication method.

The driving unit 600 rotates the sensing unit 700 of the multi-object thermal radiation measuring device 10, attached to the ceiling of the concentrated/sealed/crowded space, in the top-to-bottom direction and the side-to-side direction according to a command of any one of the other components 100, 200, 400, and 500 within the multi-object thermal radiation measuring device 10. The driving unit 600 may include the motion controller 610 and a motor 620. The sensor calibration unit 100, the object detection unit 200, the multi-object thermal radiation measuring unit 400, and the disease symptom detection unit 500 each transfer a command for rotation of the sensing unit 700 to the driving unit 600 in order to collect data, and the driving unit 600 controls the rotation of the sensing unit 700 according to the command. The command for rotation may include a vertical (top-to-bottom) or horizontal (side-to-side) rotation angle and a rotation time. The rotation angle and the rotation time, included in the command for rotation, may be replaced with a target direction and a rotational angular velocity. When the multi-object thermal radiation measuring device 10 is configured with the all-in-one design, the driving unit 600 rotates the multi-object thermal radiation measuring device 10, attached to the ceiling of the concentrated/sealed/crowded space, in the top-to-bottom direction and the side-to-side direction, according to a command of any one of the other components 100, 200, 400, and 500 within the multi-object thermal radiation measuring device 10. In this case, the contents of the command for rotation are the same as described above.

The sensing unit 700 includes a thermal sensor, generates a thermal image for the measurement target space based on a detection signal of the thermal sensor, and measures the distance to a measurement target object through the distance sensor. The sensing unit 700 may further include an RGB sensor in addition to the temperature sensor (e.g. thermal sensor) and the distance sensor (e.g. depth sensor, radar sensor, or lidar sensor). The sensing unit 700 may be installed on the ceiling of the concentrated/sealed/crowded space which is the measurement target space. The sensing unit 700 receives a data collection request from at least any one of the sensor calibration unit 100, the object detection unit 200, the multi-object thermal radiation measuring unit 400, and the disease symptom detection unit 500, and transfers sensing data (e.g. thermal image, RGB image, and distance to an object) to a component of the multi-object thermal radiation measuring device 10, which has made the request, i.e. any one of the sensor calibration unit 100, the object detection unit 200, the multi-object thermal radiation measuring unit 400, and the disease symptom detection unit 500. The sensing unit 700 generates a thermal image and RGB image on which multiple objects appear, by converting signals detected by the respective sensors included in the sensing unit 700, while the sensing unit 700 is rotated on the ceiling of the measurement target space by the driving unit 600, and measures the distances to the respective objects.

In accordance with another aspect of the present disclosure, the sensing unit 700 may be attached or connected to a portion of a structure installed in the measurement target space, not the ceiling of the measurement target space, generate the images by converting the sensor detection signals while rotated, and measure the distances to the respective objects. In this case, in order to collect information on many objects at the same time, the sensing unit 700 may be attached to as high a position as possible.

The communication unit 800 receives the metadata on the abnormal object thermal radiation precise measurement value and the abnormal object behavior detection result value from the disease symptom detection unit 500, and transmits the received metadata to the disease symptom early detection platform server 20. The communication unit 800 may serve to receive a temperature setting value from the sensor calibration unit 100 while the sensor calibration unit 100 sets the temperature of the blackbody 130, and transfer the received temperature setting value to the blackbody 130 through wired/wireless communication.

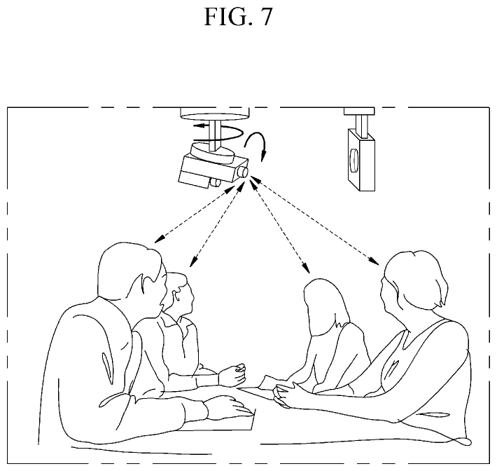

FIG. 7 is a diagram illustrating an example in which the multi-object thermal radiation measuring device in accordance with the embodiment of the present disclosure is installed and operated in a concentrated/sealed/crowded space. A respiratory syndrome such as COVID-19 usually spreads in a concentrated/sealed/crowded space such as a sanatorium, school, hospital, or meeting room. In order to block the spread of the respiratory syndrome in advance, it is more important than anything to detect the disease symptoms of multiple objects within an indoor space at the early stage, and to determine the risk of the disease, and to rapidly share the risk of the disease.

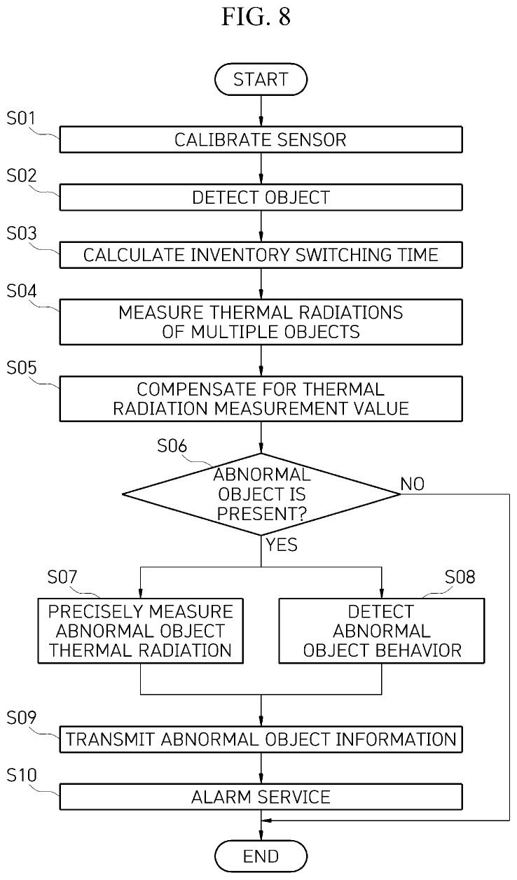

FIG. 8 is a flowchart illustrating an operating method of the multi-object thermal radiation measuring device 10 in accordance with an embodiment of the present disclosure. The operating method of the multi-object thermal radiation measuring device 10 in accordance with the embodiment of the present disclosure may include steps S01 to S10 illustrated in FIG. 8.

The multi-object thermal radiation measuring device 10 in accordance with the present disclosure is switched to the stand-by mode when power is supplied thereto. At this time, step S01 is performed.

Step S01 is a sensor calibration step. In this step, the sensor calibration unit 100 calibrates the sensors included in the multi-object thermal radiation measuring device 10. For example, the sensor calibration unit 100 calibrates the temperature sensor (e.g. thermal sensor) and the distance sensor (e.g. depth sensor) which are included in the multi-object thermal radiation measuring device 10. The sensor calibration unit 100 may use a blackbody whose temperature can be variably set, in order to calibrate the sensors. Furthermore, the sensor calibration unit 100 monitors the environment around a concentrated/sealed/crowded space, and performs the initial setting operation (temperature sensor calibration and distance sensor calibration) when the environment is changed. The sensor calibration unit 100 automatically performs the sensor calibration such that a user manipulation process such as a process of re-tuning the thermal radiation measuring device is not needed even though the environment around the concentrated/sealed/crowded space is changed. This process has been already described above with reference to FIG. 5. When the sensor calibration has been already completed and no environmental change is sensed, step S01 may be omitted.

Step S02 is an object detection step. This step includes detecting measurement target objects by applying an image recognition technology to a thermal image, and calculating the distribution of the measurement target objects according to a measurement direction. The sensing unit 700 of the multi-object thermal radiation measuring device 10, attached to the ceiling of the concentrated/sealed/crowded space, collects images (e.g. RGB image and infrared image) while rotated in the top-to-bottom direction and the side-to-side direction through the motion controller 610. The object detection unit 200 determines whether objects are present in the collected images through the image recognition technology, and calculates object distribution. At this time, the object detection unit 200 performs object detection and object tracking based on a deep learning model. The object detection unit 200 may not only detect and track one object, but also detect and track a plurality of objects (multiple objects) at the same time.

Step S03 is an inventory switching time calculation step. This step includes calculating a thermal radiation measurement time for each measurement section based on the object distribution. The dynamic switching unit 300 dynamically calculates an inventory switching time for each measurement section (zone) of the concentrated/sealed/crowded space (thermal radiation measurement time for each measurement section) based on the object detection result of the object detection unit 200. At this time, the dynamic switching unit 300 dynamically optimizes the inventory switching time according to the object distribution within the space. The dynamic switching unit 300 may set the inventory switching time of a section, in which the number of objects exceeds a predetermined reference value, to a longer time than that of a section in which the number of objects falls within the reference value, based on the object detection result. The dynamic switching unit 300 may distribute different inventory switching times depending on the number of objects to be measured, in order to efficiently perform the measurement. That is, the dynamic switching unit 300 allocates a short inventory switching time to a section (zone) having a small number of objects, and allocates a long inventory switching time to a section (zone) having a large number of objects, such that the thermal radiation measurement can be precisely performed in the section having a large number of objects.

If the multi-object thermal radiation measuring device 10 is installed in a space where the object distribution is constant, it is unnecessary to dynamically optimize the inventory switching time. Therefore, the inventory switching time for each section may be set to a specific value. In this case, steps S02 and S03 may be omitted.

Step S04 is a multi-object thermal radiation measuring step. This step includes detecting a specific portion of the face of a measurement target object by applying the image recognition technology to a thermal image, and acquiring and storing a thermal radiation measurement value of the specific portion. The multi-object thermal radiation measuring unit 400 measures the thermal radiations of multiple objects according to the inventory switching times set by the dynamic switching unit 300. The multi-object thermal radiation measuring unit 400 controls the sensing unit 700 to generate a thermal image and an RGB image while rotating the sensing unit 700 in the concentrated/sealed/crowded space in the top-to-bottom direction and the side-to-side direction through the driving unit 600, detects the face, forehead, and inner canthus of an object by applying the image recognition technology to the image, and acquires, processes, and stores the thermal radiation measurement values of the corresponding portions.

Step S05 is a thermal radiation measurement value compensation step. This step includes compensating for the thermal radiation measurement value by applying a correction value that is predetermined according to the distance to the measurement target object. The sensing unit 700 collects distance data between the distance sensor (e.g. radar sensor, depth sensor, or lidar sensor) and the measurement target object through the distance sensor, and transfers the collected distance data to the multi-object thermal radiation measuring unit 400. The thermal radiation measurement value compensation module 410 included in the multi-object thermal radiation measuring unit 400 compensates for the thermal radiation measurement value based on data on the distance to the object or the forehead or inner canthus of the object. That is, the thermal radiation measurement value compensation module 410 compensates for the thermal radiation measurement value by applying a correction value that is predetermined according to the distance. Step S05 may be omitted depending on the characteristics of the space. For example, when the multi-object thermal radiation measuring device 10 is installed on the ceiling of a narrow space, step S05 may be omitted because the distances between the multi-object thermal radiation measuring device 10 and the respective objects have small deviations.

Step S06 is to determine whether an abnormal object is present. This step includes determining whether each measurement target object is abnormal, according to a predetermined criterion based on the thermal radiation measurement value. The object state determination module 510 of the disease symptom detection unit 500 determines whether each object is normal or abnormal, based on the multi-object thermal radiation measurement result. In the present disclosure, an object that is determined to be abnormal by the object state determination module 510, i.e. an object having a symptom, is referred to as an abnormal object. When an abnormal object is present, steps S07 and S08 are performed, and when no abnormal object is present in the entire sections within the space, the procedure is ended.

Step S07 is an abnormal object thermal radiation measurement step. This step includes precisely measuring the thermal radiation of the abnormal object based on the thermal image in which the abnormal object appears. The abnormal object region detection module 520 detects a region in which an object having a high fever symptom is present, by using the image recognition technology. The abnormal object thermal radiation precise measurement module 530 performs precise thermal radiation measurement on the region in which the abnormal object is present.

Step S08 is an abnormal object behavior detection step. The abnormal object behavior detection module 540 detects the behavior of the abnormal object in the region in which the abnormal object is present. For example, the abnormal object behavior detection module 540 detects an abnormal behavior related to the respiratory system, such as cough, sneeze, or respiratory difficulty of the abnormal object. The abnormal object behavior detection module 540 may utilize a deep learning-based behavior detection model in order to detect the disease symptoms of the abnormal object at the early stage. For example, the abnormal object behavior detection module 540 may track the location of an object who coughs or sneezes and the number of times that the object coughs or sneezes, by using a cough/sneeze recognition model generated through a supervised learning technique based on the CNN. When steps S07 and S08 are completed, step S09 is performed. Step S08 may be omitted in some cases. That is, in step S09, only metadata on the object thermal radiation measurement result may be transmitted.

Step S09 is an abnormal object information transmission step. This step includes detecting the disease symptom of the abnormal object based on at least any one of the thermal radiation precise measurement result and the abnormal object behavior detection result, and transmitting metadata on the thermal radiation precise measurement value and the behavior detection result and the disease symptom detection result to the disease symptom early detection platform server. The abnormal object thermal radiation precise measurement value and the abnormal object behavior detection result value are transmitted as the metadata to the disease symptom early detection platform server 20 through the communication unit 800.

Step S10 is an alarm service step. The disease symptom early detection platform server 20 is a cloud-based service platform, and serves to monitor data collected by the multi-object thermal radiation measuring devices 10 installed in main concentrated/sealed/crowded spaces across the country, evaluate the risk of the disease, and perform service management. The disease symptom early detection platform server 20 may calculate the disease risk based on the metadata on the thermal radiation precise measurement values, the metadata on the abnormal object behavior detection result values, and the disease symptom detection results of the abnormal objects, which are collected through nationwide networks, and provide an alarm service to inform the whole nation of the disease risk and the statistics of the disease symptom detection results through at least any one communication method of a wired communication method and a wireless communication method. Steps S09 and S10 may be omitted. For example, immediate preventive measures may be made on the basis of only the abnormal object thermal radiation precise measurement result and the abnormal object behavior detection result, which are obtained through steps S07 and S08.

For reference, the components in accordance with the embodiment of the present disclosure may be each implemented in software or implemented as a hardware component such as DSP (Digital Signal Processor), FPGA (Field Programmable Gate Array) or ASIC (Application Specific Integrated Circuit), and play predetermined roles.

However, the meaning of ‘components’ is not limited to software or hardware, but each component may be configured in an addressable storage medium or configured to execute one or more processors.

Therefore, for example, the components include components such as software components, object-oriented software components, class components, and task components. Furthermore, the components include processes, functions, attributes, procedures, subroutines, segments of a program code, drivers, firmware, microcode, circuit, data, database, data structures, tables, arrays, and variables.

The components and functions provided in the corresponding components may be combined into a smaller number of components or further separated into additional components.

In this case, it may be understood that blocks of flowcharts and combinations of the flowcharts can be executed by computer program instructions. Since the computer program instructions can be mounted on a processor of a general-purpose computer, a special computer, or other programmable data processing equipment, the instructions executed through the processor of the computer or the programmable data processing equipment generate means for executing functions described in the flowchart blocks. The computer program instructions can implement functions through a specific method by using a computer or other programmable data processing equipment, or can be stored in a computer readable memory. Therefore, the instructions using the computer or stored in the computer readable memory can be used to fabricate a product including instruction means for executing the functions described in the flowchart blocks. Since the computer program instructions can be mounted on a computer or other programmable data processing equipment, instructions that generates processes to be executed by the computer while a series of operation steps are executed on the computer or other programmable data processing equipment, and operates the computer or other programmable data processing equipment may provide steps for executing the functions described in the flowchart blocks.

Furthermore, each of the blocks may indicate a module, a segment, or a part of a code, which includes one or more executable instructions for executing specific logical functions. In some alterative examples, it should be noted that the functions described in the blocks can be executed out of order. For example, two blocks which are successively illustrated can be executed substantially at the same time, or can be executed sometimes in the reverse order according to the corresponding function.

The term ‘ . . . unit’ or ‘module’ used in the present embodiment indicates software or a hardware component such as FPGA or ASIC, and ‘ . . . unit’ or ‘module’ plays a certain role. However, the meaning of ‘ . . . unit’ or ‘module’ is not limited to software or hardware. That is, ‘ . . . unit’ or ‘module’ may be configured in an addressable storage medium, or configured to execute one or more processors. Therefore, for example, ‘ . . . unit’ or ‘module’ includes components such as software components, object-oriented software components, class components, and task components. Furthermore, ‘ . . . unit’ or ‘module’ includes processes, functions, attributes, procedures, subroutines, segments of a program code, drivers, firmware, microcode, circuit, data, database, data structures, tables, arrays, and variables. Functions provided in a plurality of components, ‘ . . . unit’, or ‘module’ may be combined into a smaller number of components, ‘ . . . units’, or ‘modules’, or further separated into additional components, ‘ . . . units’, or ‘modules’. Furthermore, the components, ‘ . . . units’, and ‘modules’ may be implemented to execute one or more CPUs within a device or security multimedia card.

The operating method of the multi-object thermal radiation measuring device has been described with reference to the flowchart illustrated in FIG. 8. Although the operating method has been illustrated as a series of blocks for convenience of description, the present disclosure is not limited to the sequence of the blocks, but some blocks may be executed in a different order from that illustrated and described in this specification or executed at the same time as other blocks, and various branches, flow paths, and block orders may be implemented to accomplish the same or similar result. For example, a combination of specific blocks may be repeatedly executed. Furthermore, all of the blocks which are illustrated to implement the method described in this specification may not be required. For example, at least any one step of steps S01, S02, S03, S05, S08, S09, and S10, which have been described with reference to FIG. 8, may be omitted.

From the foregoing, the configurations of the present disclosure have been described in detail with reference to the accompanying drawings, but are illustrative only. It should be understood that the person skilled in the art can variously modify and change the present disclosure without departing from the technical spirit of the present disclosure. Therefore, the scope of the present disclosure is defined by the claims to be described below rather than the detailed description, and it should be construed that all the changed and modified forms derived from the claims and the equivalent concept thereof fall within the technical scope of the present disclosure.

What is claimed is:

1. A multi-object thermal radiation measuring device comprising:
a sensing unit comprising a thermal sensor, and configured to generate a thermal image for a measurement target space based on a detection signal of the thermal sensor;
a multi-object thermal radiation measuring unit configured to detect a specific portion of a measurement target object by applying an image recognition technology to the thermal image, and acquire and store a thermal radiation measurement value of the specific portion;
a disease symptom detection unit configured to determine whether each measurement target object is abnormal, according to a predetermined criterion based on the thermal radiation measurement value, and detect a disease symptom of an abnormal object based on a result obtained by precisely measuring the thermal radiation of the abnormal object; and
a driving unit configured to rotate the sensing unit according to a command transferred by any one of the multi-object thermal radiation measuring unit and the disease symptom detection unit.

2. The multi-object thermal radiation measuring device of claim 1, wherein the sensing unit is installed on the ceiling of the measurement target space.

3. The multi-object thermal radiation measuring device of claim 1, wherein the sensing unit comprises a distance sensor, and measures a distance to the measurement target object through the distance sensor,
wherein the multi-object thermal radiation measuring unit compensates for the thermal radiation measurement value by applying a correction value that is predetermined according to the distance to the measurement target object.

4. The multi-object thermal radiation measuring device of claim 1, further comprising a sensor calibration unit configured to calibrate a sensor included in the sensing unit,
wherein the sensing unit comprises a distance sensor,
the sensor calibration unit is installed at a predetermined distance from the sensing unit, and calibrates the sensor by using a blackbody whose temperature is variably set by the sensor calibration unit, and
the driving unit rotates the sensing unit according to a command of the sensor calibration unit, such that the sensing unit faces the blackbody.

5. The multi-object thermal radiation measuring device of claim 4, wherein the sensing unit and the blackbody are installed on the ceiling of the measurement target space.

6. The multi-object thermal radiation measuring device of claim 1, further comprising:
an object detection unit configured to detect the measurement target object by applying an image recognition technology to the thermal image, and calculate the distribution of measurement target objects according to a measurement direction; and
a dynamic switching unit configured to determine a thermal radiation measurement time for each measurement section based on the distribution,
wherein the multi-object thermal radiation measuring unit generates a command for rotation of the sensing unit based on the thermal radiation measurement time for each measurement section,
the driving unit rotates the sensing unit according to the command of the object detection unit, and
the measurement section is configured by dividing the measurement target space according to a predetermined criterion based on the rotatable range of the sensing unit.

7. The multi-object thermal radiation measuring device of claim 1, wherein the specific portion is at least any one of the forehead and inner canthus of the measurement target object.

8. The multi-object thermal radiation measuring device of claim 1, wherein the disease symptom detection unit extracts a spatial coordinate of the abnormal object from a thermal image in which the abnormal object appears, generates a command for rotation of the sensing unit based on the spatial coordinate and a tracking result for the abnormal object, transfers the command to the driving unit, receives, from the sensing unit, an additional thermal image in which the abnormal object appears, and precisely measures the thermal radiation of the abnormal object based on the additional thermal image.

9. The multi-object thermal radiation measuring device of claim 1, wherein the disease symptom detection unit detects the behavior of the abnormal object by utilizing a deep learning-based behavior detection model, and detects a disease symptom of the abnormal object based on at least any one of the thermal radiation precise measurement result and the behavior detection result.

10. The multi-object thermal radiation measuring device of claim 1, further comprising a communication unit,
wherein the disease symptom detection unit generates metadata on the thermal radiation precise measurement value, and
the communication unit transmits the metadata to an information system which is present outside.

11. A disease risk warning system comprising:
the multi-object thermal radiation measuring device of claim 10, and
a disease symptom early detection platform server configured to calculate a disease risk based on the disease symptom detection result and the metadata collected by the multi-object thermal radiation measuring device, and provide an alarm service for the disease risk by using at least any one communication method of a wired communication method and a wireless communication method.

12. An operating method of a multi-object thermal radiation measuring device, comprising:
a multi-object thermal radiation measuring step of detecting a specific portion of a measurement target object by applying an image recognition technology to a thermal image, and acquiring and storing a thermal radiation measurement value of the specific portion;
an abnormal object determination step of determining whether each measurement target object is abnormal, according to a predetermined criterion based on the thermal radiation measurement value;
an abnormal object thermal radiation precise measurement step of precisely measuring the thermal radiation of an abnormal object based on a thermal image in which the abnormal object appears; and
an abnormal object information transmitting step of detecting a disease symptom of the abnormal object based on the thermal radiation measurement result, and transmitting metadata on the thermal radiation precise measurement value and the disease symptom detection result to a disease symptom early detection platform server.

13. The operating method of claim 12, further comprising a sensor calibration step of calibrating a sensor included in the multi-object thermal radiation measuring device, before the multi-object thermal radiation measuring step.

14. The operating method of claim 12, further comprising, before the multi-object thermal radiation measuring step:
an object detection step of detecting the measurement target object by applying the image recognition technology to the thermal image, and calculating the distribution of measurement target objects according to a measurement direction; and
an inventory switching time calculation step of calculating a thermal radiation measurement time for each measurement section based on the distribution.

15. The operating method of claim 12, further comprising, after the multi-object thermal radiation measuring step, a thermal radiation measurement value compensation step of compensating for the thermal radiation measurement value by applying a correction value that is predetermined according to a distance to the measurement target object.

16. The operating method of claim 12, further comprising, after the abnormal object thermal radiation precise measurement step, an abnormal object behavior detection step of detecting the behavior of the abnormal object by utilizing a deep learning-based behavior detection model.

17. The operating method of claim 12, further comprising, after the abnormal object information transmitting step, an alarm service step of calculating a disease risk based on the metadata and the disease symptom detection result, and providing an alarm service for the disease risk by using at least any one communication method of a wired communication method and a wireless communication method.

18. The operating method of claim 12, wherein the specific portion of the measurement target object is at least any one of the forehead and inner canthus of the measurement target object.

19. The operating method of claim 13, wherein the sensor calibration step comprises calibrating the sensor by using a blackbody whose temperature is variably set.

20. The operating method of claim 16, wherein the abnormal object behavior detection step comprises detecting whether the abnormal object has at least any one of cough and sneeze, by utilizing a deep learning-based behavior detection model.
